SEQUENCE LISTING (33 sequences)

SEQ ID NO: 1 - DNA, 19 bp, Artificial Sequence
  primer
cggcctcgctaaccaacat

SEQ ID NO: 2 - DNA, 23 bp, Artificial Sequence
  primer
cccaaagtaaatccacagcattt

SEQ ID NO: 3 - DNA, 19 bp, Artificial Sequence
  primer
atggtctaaatacaacttc

SEQ ID NO: 4 - DNA, 19 bp, Artificial Sequence
  primer
gagtcggaggagcgtggtt

SEQ ID NO: 5 - DNA, 17 bp, Artificial Sequence
  primer
cggcaccgtcaccttga

SEQ ID NO: 6 - DNA, 19 bp, Artificial Sequence
  primer
caagcctacttaaatggaa

SEQ ID NO: 7 - DNA, 22 bp, Artificial Sequence
  primer
aaaatcagggtagacgcaatcc

SEQ ID NO: 8 - DNA, 22 bp, Artificial Sequence
  primer
gaacagagcaatcccaaaacct

SEQ ID NO: 9 - DNA, 16 bp, Artificial Sequence
  primer
acctcagctgaaccaa

SEQ ID NO: 10 - DNA, 20 bp, Artificial Sequence
  primer
ctgcattgctgcctcttctg

SEQ ID NO: 11 - DNA, 17 bp, Artificial Sequence
  primer
ccgcatccgttcaccaa

SEQ ID NO: 12 - DNA, 17 bp, Artificial Sequence
  primer
aaacgtaggcggagtca

SEQ ID NO: 13 - DNA, 19 bp, Artificial Sequence
  primer
cagcgctttcagcaattgc

SEQ ID NO: 14 - DNA, 20 bp, Artificial Sequence
  primer
gaycggagcgatgcatagtc

SEQ ID NO: 15 - DNA, 15 bp, Artificial Sequence
  primer
cccatatgacatccc

SEQ ID NO: 16 - DNA, 20 bp, Artificial Sequence
  primer
gtaatgcacctaaagggcac

SEQ ID NO: 17 - DNA, 22 bp, Artificial Sequence
  primer
cctccttcactycttactgraa

SEQ ID NO: 18 - DNA, 21 bp, Artificial Sequence
  primer
gcaacaggtttcagrggaaga

SEQ ID NO: 19 - DNA, 19 bp, Artificial Sequence
  primer
gtraacactgctctcggct

SEQ ID NO: 20 - DNA, 20 bp, Artificial Sequence
  primer
yatgaggaactgcggaggag

SEQ ID NO: 21 - DNA, 20 bp, Artificial Sequence
  primer
gctgttgcaaccattgacac

SEQ ID NO: 22 - DNA, 20 bp, Artificial Sequence
  primer
tacacagrgcgacmacaacs

SEQ ID NO: 23 - DNA, 20 bp, Artificial Sequence
  primer
cayrctycccacaacacccc

SEQ ID NO: 24 - DNA, 17 bp, Artificial Sequence
  primer
acaccgatcagcacatg

SEQ ID NO: 25 - DNA, 20 bp, Artificial Sequence
  primer
tggccctcctctgtaaggaa

SEQ ID NO: 26 - DNA, 20 bp, Artificial Sequence
  primer
ggagtgctcctggaggtgta

SEQ ID NO: 27 - DNA, 20 bp, Artificial Sequence
  primer
cctctggtggacatcctctg

SEQ ID NO: 28 - DNA, 34 bp, Artificial Sequence
  primer
agcgctgaagtatccgcatccgttcaccaagttg

SEQ ID NO: 29 - DNA, 26 bp, Artificial Sequence
  primer
accaaccttcgtgggagcagctgagc

SEQ ID NO: 30 - DNA, 20 bp, Artificial Sequence
  primer
gycttactgaraygggrtca

SEQ ID NO: 31 - DNA, 20 bp, Artificial Sequence
  primer
tgagrtascakcckccccag

SEQ ID NO: 32 - DNA, 25 bp, Artificial Sequence
  primer
cgacgatgactctctactgtgtgat

SEQ ID NO: 33 - DNA, 20 bp, Artificial Sequence
  primer
tgcatcctgctgygyagcat

(19) United States
(12) Patent Application Publication
(10) Pub. No.: US 2023/0174996 A1
(43) Pub. Date: Jun. 8, 2023

(54) REASSORTED ISA VIRUS

(71) Applicant: Intervet Inc., Madison, NJ (US)

(72) Inventors: Stephane Villoing, Ulset (NO); Sven Leininger, Bergen (NO); Kjartan Hodneland, Bergen (NO); Joseph Koumans, Wageningen (NL)

(73) Assignee: Intervet Inc., Madison, NJ (US)

(21) Appl. No.: 17/785,223

(22) PCT Filed: Dec. 18, 2020
(86) PCT No.: PCT/EP2020/086935

§ 371 Date: Jun. 14, 2022

(30) Foreign Application Priority Data
Dec. 20, 2019 (EP) 19218730.0

(51) Int. Cl. C12N 15/63; C12N 7/02

(52) U.S. Cl. CPC C12N 15/63; C12N 7/02; C12N 2760/16021; C12N 2760/16034; C12N 2760/16051

(57) ABSTRACT

The present invention is directed to a reassorted ISA virus comprising Genome segments 1-8 wherein at least one genome segment is from Genotype I and at least one genome segment is from genotype II, wherein genome segment 6 is of Genotype II. The reassorted ISA virus was found to grow well in suspension culture. The present invention is also directed to method to make the reassorted virus as well as vaccination methods using the reassorted ISA virus and to vaccine compositions comprising the reassorted ISA virus.

### TECHNICAL FIELD

This invention is in the field of ISA virus reassortment. Furthermore, it relates to manufacturing vaccines for protecting against ISA viruses.

### BACKGROUND ART

Infectious salmon anemia (ISA) is a viral disease of Atlantic salmon (Salmo salar) caused by Salmon isavirus. It affects fish farms in Canada, Norway, Scotland and Chile, causing severe losses to infected farms. The disease is caused by a general infection which among others cause severe anaemia and bleeding lesions. The disease spreads slowly in an infected fish farm, and the mortality can vary from 15-100%.

The ISA virus (ISAV) contains a negatively charged single-stranded RNA genome of 8 segments. The total size of the segments is 14.5 Kb (1.5 × 103 base pairs). The virus replicates itself in the nucleus. It is a 100-120 nm enveloped virus with 10 nm peplomers, and it separates itself from the cell membrane by budding. The virus belongs to the family Orthomyxoviridae,. The genome encodes at least 10 proteins. Sequence analysis of the segments from different ISAV consistently reveal two genotypes designated according to their geographic origin as European (genotype l) and North American (Genotype ll).

The first ISA outbreak in Chile occurred from mid-June 2007 to 2010 and is now endemic disease in Chile. Chile has after Norway the largest production of Atlantic Salmon. The ISAV isolates found in Chile are from the European genotype l, and have likely arisen from a Norwegian source.

Vaccination is a measure to control ISA infections. Commercial ISAV vaccines are available, based on injection of oil-adjuvanted, inactivated ISAV cell culture antigens, as well vaccines based on ISAV HE-protein expressed in yeast.

SHK-1, TO, ASK-2 and CHSE-214 are commonly employed cell lines for ISAV isolation, these cell lines also have limitations. The former three cell lines require very low split ratios and the latter cell line does not support the growth of all ISAV isolates. CPE development may take up to 17 days in CHSE-214 cells with a virus yield lower than SHK-1. SHK-1 is a very delicate cell line requiring complex growth medium and sometimes, it loses sensitivity at higher passages, moreover, some ISAV isolates produce poorly defined and slowly developing CPE in the SHK-1 cell line (Munir 2006, J. Vet. Sci 7(2), p167-176). There appears to be a geographical correlation with the ability to grow in CHSE cells although the molecular basis for this is not known (Kibbenge et al, 2005, Vir. Journal 2, (75), p1-6). In general, North American isolates, genotype II, usually grow on CHSE-214 cells whereas the European isolates, genotype I, usually grow less well or not at all on CHSE-214 cells. Chile ISAV isolates belong to European Genotype I and do also not grow well on CHSE-214 cells.

Hence there is need for improved culturing of the ISAV, especially ISAV from genotype I, more especially Chile ISAV. Preferably there is an increased yield of ISAV.

There are basically two systems for growing virus in cell cultures, as monolayers on an artificial substrate (i.e., adherent culture) or free-floating in the culture medium (suspension culture). Suspension cell culture has advantages over adherent cell culture, such as easier to passage, does not require enzymatic or mechanical dislocation, is more easy to scale up and can be maintained in culture vessels. Hence it would be advantageous to be able to grow ISAV in suspension cells. Unfortunately, Chile type ISA typically grows very slow in cell culture, especially in suspension cell culture.

It is also advantageous to be able to grow virus on culture cells in serum free medium.

### SUMMARY OF INVENTION

Surprisingly it was found that a reassorted ISA virus comprising genome segment 1-8, wherein at least one genome segment is from Genotype I and at least one genome segment is from genotype II, wherein genome segment 6 is of Genotype II grows well on suspension cells in serum free medium and provides protection against infection with ISAV.

In one embodiment of the invention and/or embodiments thereof, the reassorted ISA virus comprises at two least genome segments of Genotype I.

In one embodiment of the invention and/or embodiments thereof, the reassorted ISA virus comprises a genome segment 5 of Genotype I.

In one embodiment of the invention and/or embodiments thereof, the reassorted ISA virus comprises a genome segment 8 of Genotype I.

In one embodiment of the invention and/or embodiments thereof, the reassorted ISA virus comprises at least one genome segment of Genotype I that is of Chilean origin.

In one embodiment of the invention and/or embodiments thereof, the reassorted ISA virus comprises at least one genome segment of Genotype II that is of Canadian origin.

Another aspect of the invention and/or embodiments thereof, is directed to a vaccine composition comprising a reassorted ISA virus according to any embodiment as described herein.

Another aspect of the invention and/or embodiments thereof, is directed to a method to produce reassorted ISA virus according to any embodiment as described herein comprising the steps of (i) infecting a culture host with an ISA virus strain of genotype I and an ISA virus strain of genotype II;
(ii) culturing the culture host in order to produce reassorted ISAV virus and
(iii) determine if the reassorted ISAV virus has a genome segment 6 of genotype II present and at least one other genome segment of genotype I.

In one embodiment of the invention and/or embodiments thereof, the method comprises a further step of (iv) purifying the virus obtained in step (iii).

Another aspect of the invention and/or embodiments thereof, is directed to a method to culture reassorted ISA comprising
(i)infecting a culture host with the reassorted virus according to any embodiment as described herein
(ii) culturing the host from step (i) to produce further virus;

In one embodiment of the invention and/or embodiments thereof, the method to produce a reassorted ISA virus further comprises a step (iii) purifying virus obtained in step (ii).

In one embodiment of the invention and/or embodiments thereof, the culture host is a suspension cell.

In one embodiment of the invention and/or embodiments thereof, the culture host is a CHSE-214 cell.

Another aspect of the invention and/or embodiments thereof, is directed to a reassorted ISA virus according to any embodiment as described herein for use in a method to vaccinate fish against ISA virus .

### DETAILED DESCRIPTION

### Definitions

As used in the specification and the appended claims the term “treatment” is to be understood as bringing a body from a pathological state back to its normal, healthy state or preventing a pathological state. The latter may be denoted as “prophylactic treatment”. Treatment is meant to cover protection against a pathological state. Treatment also means to have a reduction in pathological changes when compared to individuals that have not been treated. Suitably, there is at least a reduction of 10% in pathological changes, more preferably, at least a reduction of 25%, 20%, 25%, 30%, 40%, 50%, 60%, 65%, 70%, 75%, 80%, 85%, 90%, 95% or even a 100% in pathological changes when compared to individuals that have not been treated.

As used in the specification and the appended claims the term “provides protection” is to be understood as preventing a pathological state, such as “prophylactic treatment”. Protection also means to have less pathological changes when compared to individuals that have not been treated. Suitably, there is at least 10% less pathological changes, more preferably, at least 25%, 20%, 25%, 30%, 40%, 50%, 60%, 65%, 70%, 75%, 80%, 85%, 90%, 95% or even a 100% less pathological changes when compared to individuals that have not been treated.

The term “pharmaceutically acceptable carrier” is intended to include formulation used to stabilize, solubilize and otherwise be mixed with active ingredients to be administered to living animals, including fish. This includes any and all solvents, dispersion media, coatings, antibacterial and antifungal agents, isotonic and absorption delaying agents, and the like, compatible with pharmaceutical administration.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

The ISAV is commonly indicated into two genotypes. These genotypes are being named genotype I and genotype II. Sometimes these genotypes are being referred to European genotype (genotype I) and North American (genotype II). The genotyping is based on sequence analysis of the segments from different ISAV. These cluster into two different and distinguishable gentoypes (Kibenge et al. (2001) Antigenic variation among isolates of infectious salmon anaemia virus correlates with genetic variation of the viral haemagglutinin gene. J Gen Virol 82:2869-2879; Kibenge et al. Discovery of variant infectious salmon anaemia virus (ISAV) of European genotype in British Columbia, Canada. Virol J 13, 3 (2016)) The EU group has been further divided into four clades (genogroups): three European (EU-1 to EU-3) and a European-like group from north- eastern North America (EU-NA).

Reassorted ISA virus were obtained comprising at least one genome segment from Genotype I and at least one genome segment from genotype II. It was found that when genome segment 6 is of Genotype II, the ISA virus grows well on suspension cells and in serum free medium. This was the first time that reassorted ISA viruses were obtained. As discussed above, the ISA virus genome contains eight segments. The words “segment” and “genome segment” or “genomic segment” are used interchangeably and are all directed to one or more of the eight segments of the ISA virus genome.

Preferably, the reassorted ISA virus comprises at two least segments of Genotype II, more preferably, at least 2-7 segments are of Genotype II, more preferably at least 3-6 segments are of Genotype II, more preferably at least 4-5 segments are of Genotype II. In a preferred embodiment of the invention and/or embodiments thereof the ISA virus comprises 2-6 segments from Genotype II, more preferably, 2-5 segment from Genotype II.

It was found that reassorted ISA viruses wherein genome segment 6 is of Genotype II, the ISA virus grow well on suspension cells and in serum free medium. In a preferred embodiment, at least segment 6 and 1 are Genotype II. In a preferred embodiment of the invention and/or embodiments thereof, at least segment 6 and 2 are Genotype II. In a preferred embodiment of the invention and/or embodiments thereof, at least segment 6 and 3 are Genotype II. In a preferred embodiment, at least segment 6 and 4 are Genotype II. In a preferred embodiment, at least segment 6 and 7 are Genotype II. In a preferred embodiment of the invention and/or embodiments thereof, at least segment 6 and at least two genome segments selected of the of the group comprising segment 1, segment, 2, segment, 3, segment 4, segment 7 and segment 8 are Genotype II. In a preferred embodiment of the invention and/or embodiments thereof, at least segment 6 and at least two genome segments selected of the of the group comprising segment 1, segment, 2, segment, 3, and segment 4, are Genotype II. In a preferred embodiment of the invention and/or embodiments thereof, at least segment 6 and at least three genome segments selected of the of the group comprising segment 1, segment, 2, segment, 3, segment 4, segment 7 and segment 8 are Genotype II. In a preferred embodiment of the invention and/or embodiments thereof, at least segment 6, and at least three genome segments selected of the of the group comprising segment 1, segment, 2, segment, 3, and segment 4, are Genotype II.

At least one genome segment should be of Genotype I to enable protection or treatment against an infection with a ISA virus, preferably a ISA virus of genotype I. Preferably, the reassorted ISA virus comprises at two least segments are of Genotype I, more preferably, at least 2-7 segments are of Genotype I, more preferably at least 3-6 segments are of Genotype I, more preferably at least 3-6 segments are of Genotype I, more preferably at least 4-5 segments are of Genotype I. In a preferred embodiment of the invention and/or embodiments thereof the ISA virus comprises 2-6 segments from Genotype I, more preferably, the ISA virus comprises 2-5 segments from Genotype I.

In a preferred embodiment of the invention and/or embodiments thereof, at least segment 5 is from Genotype I. In a preferred embodiment of the invention and/or embodiments thereof, at least segment 8 is Genotype I. In a preferred embodiment of the invention and/or embodiments thereof, at least segment 7 is Genotype I. In a preferred embodiment of the invention and/or embodiments thereof, at least segment 3 is Genotype I. In a preferred embodiment of the invention and/or embodiments thereof, at least segment 5 and 8 are Genotype I. In a preferred embodiment of the invention and/or embodiments thereof, at least segment 5 and 7 are Genotype I. In a preferred embodiment of the invention and/or embodiments thereof, at least segment 5 and 3 are Genotype I. In a preferred embodiment of the invention and/or embodiments thereof, at least segment 7 and 8 are Genotype I. In a preferred embodiment of the invention and/or embodiments thereof, at least segment 3 and 7 are Genotype I. In a preferred embodiment of the invention and/or embodiments thereof, at least segment 3 and 8 are Genotype I. In a preferred embodiment of the invention and/or embodiments thereof, at least segment 5, 7 and 8 are Genotype I. In a preferred embodiment of the invention and/or embodiments thereof, at least segment 5, 7 and 3 are Genotype I. In a preferred embodiment of the invention and/or embodiments thereof, at least segment 5, 3 and 8 are Genotype I. In a preferred embodiment of the invention and/or embodiments thereof, at least segment 3, 5, 7 and 8 are Genotype I.

In a preferred embodiment of the invention and embodiments thereof, the reassorted virus provides protection against infection of an ISA virus of genotype I. In a preferred embodiment of the invention and embodiments thereof, the reassorted virus provides protection against infection of an ISA virus of genotype II. In a preferred embodiment of the invention and embodiments thereof, the reassorted virus provides protection against infection of an ISA virus of genotype I and Genotype II.

In one embodiment of the invention and/or embodiments thereof, the reassorted ISA virus comprises at least one genome segment of Genotype I that is of Chilean origin. The segment of Chilean origin means that the segment is derived from a Chilean strain. In one embodiment of the invention and/or embodiments thereof, the reassorted ISA virus comprises at least two genome segments of Genotype I that are of Chilean origin. In one embodiment of the invention and/or embodiments thereof, the reassorted ISA virus comprises at least three genome segments of Genotype I that are of Chilean origin. In one embodiment of the invention and/or embodiments thereof, the reassorted ISA virus comprises at least four segments of Genotype I that is of Chilean origin.

In one embodiment of the invention and/or embodiments thereof, the reassorted ISA virus comprises at least one genome segment of Genotype II that is of Canadian origin. The segment of Canadian origin means that the segment is derived from a Canadian strain. In one embodiment of the invention and/or embodiments thereof, the reassorted ISA virus comprises at least two genome segments of Genotype II that are of Canadian origin. In one embodiment of the invention and/or embodiments thereof, the reassorted ISA virus comprises at least three genome segments of Genotype II that are of Canadian origin. In one embodiment of the invention and/or embodiments thereof, the reassorted ISA virus comprises at least four segments of Genotype II that are of Canadian origin.

The Genomic organization of ISA virus is known (see e.g. Clouthier et al J. Gen. Virol 2002; 421-428)

Eight segments of genomic viral RNA between 1.0 and 2.4 kb in length were identified.

Each segment encodes the following proteins

| Segment | Encoded protein |
|---|---|
| 1 | Polymerase (PB2) |
| 2 | Polymerase (PB1) |
| 3 | Nucleoprotein (NP) |
| 4 | RNA polymerase (PA) |
| 5 | Fusion (F) |
| 6 | Hemagglutinin-esterase (HE) |
| 7 | nonstructural proteins (NS1, NS2) |
| 8 | Matrix proteins (M1, M2) |

The genotype and origin of the genome segment can be easily determined by RT-PCR and/or phylogenetic analysis, see e.g. the experimental section. Segments of ISAV, including its genotype and origin, have been determined several times (see e.g. Kibenge et al. 2001, J. Gen Virol, 82, p2869-2879; Kibenge et al. 2016 Virology Journal 13:3; Cottet et al. J. Virol, 2010, p11916-11928). In here primers and probes are disclosed for the different segments. Phylogentic analysis provides information on the regional origin and/or genotype by aligning sequences by methods well know such as Clustal X and then generating phylogenetic trees by well-known methods such as Clustal X.

The sequence of each segment of ISAV can be determined and then compared to other ISAV sequences known (e.g. in Genbank) and classified as a certain genotype, using e.g. BLASTN. Members of the same genotype have 90% similarity or higher whereas members of two different genotypes have a similarity lower than 90%, lower than 85% and even lower than 80%. It was found that especially segments 2, 6, and 8, provide the best determination of the genotype. Furthermore, using only the extracellular region of hemagglutinin, the European isolates can be divided into subgroups (Cottet et al. Virus Research 155 (2011) 10-19).

For example, using primer SEQ ID NO: 10 (forward primer genotype II segment 6) and primer SEQ ID NO: 11 (reverse primer genotype II segment 6) one obtains a segment 6 amplicon. If this segment is a genotype II then this amplicon has at least 90% sequence identity with other segments 6 of genotype II ISAV or has less than 85%, or even less than 80% sequence identity with other segments 6 of genotype I ISAV.

For example, using primer SEQ ID NO: 7 (forward primer genotype I segment 6) and primer SEQ ID NO: 8 (reverse primer genotype I segment 6) one obtains a segment 6 amplicon. If this segment is a genotype I then this amplicon has at least 90% sequence identity with other segments 6 of genotype I ISAV or has less than 85%, or even less than 80% sequence identity with other segments 6 of genotype II ISAV.

In an embodiment of the present invention and/or embodiments thereof, the genotype of segment 6 is determined by SEQ ID NO: 7 and 8 and by SEQ ID NO: 10, and 11. If the amplicon produced by these primers has more than 90% identity with segment 6 of genotype II ISAV then the segment 6 is genotype II.. If the amplicon produced by these primers has more than 90% identity with segment 6 of genotype I ISAV then the segment 6 is genotype I.

Suitable comparison of the segment 6 can be made for genotype II for example with NC_006499.

Also for the other segments the genotype can be determined.

### Segment 1

For example, using primer SEQ ID NO: 16 (forward primer segment 1) and primer SEQ ID NO: 17 (reverse primer segment 1) one obtains a segment 1 amplicon. If this segment is a genotype II then this amplicon has at least 90% sequence identity with other segments 1 of genotype II ISAV or has less than 85%, or even less than 80% sequence identity with other segments 1 of genotype I ISAV. If this segment is a genotype I then this amplicon has at least 90% sequence identity with other segments 1 of genotype I ISAV or has less than 85%, or even less than 80% sequence identity with other segments 1 of genotype II ISAV.

### Segment 2

For example, using primer SEQ ID NO: 18 (forward primer segment 2) and primer SEQ ID NO: 19 (reverse primer segment 2) one obtains a segment 2 amplicon. If this segment is a genotype II then this amplicon has at least 90% sequence identity with other segments 2 of genotype II ISAV or has less than 85%, or even less than 80% sequence identity with other segments 2 of genotype I ISAV. If this segment is a genotype I then this amplicon has at least 90% sequence identity with other segments 2 of genotype I ISAV or has less than 85%, or even less than 80% sequence identity with other segments 2 of genotype II ISAV.

### Segment 3

For example, using primer SEQ ID NO: 20 (forward primer segment 3) and primer SEQ ID NO: 21 (reverse primer segment 3) one obtains a segment 3 amplicon. If this segment is a genotype II then this amplicon has at least 90% sequence identity with other segments 3 of genotype II ISAV or has less than 85%, or even less than 80% sequence identity with other segments 3 of genotype I ISAV. If this segment is a genotype I then this amplicon has at least 90% sequence identity with other segments 3 of genotype I ISAV or has less than 85%, or even less than 80% sequence identity with other segments 3 of genotype II ISAV.

### Segment 4

For example, using primer SEQ ID NO: 22 (forward primer segment 4) and primer SEQ ID NO: 23 (reverse primer segment 4) one obtains a segment 4 amplicon. If this segment is a genotype II then this amplicon has at least 90% sequence identity with other segments 4 of genotype II ISAV or has less than 85%, or even less than 80% sequence identity with other segments 4 of genotype I ISAV. If this segment is a genotype I then this amplicon has at least 90% sequence identity with other segments 4 of genotype I ISAV or has less than 85%, or even less than 80% sequence identity with other segments 4 of genotype II ISAV.

### Segment 4

For example, using primer SEQ ID NO: 24 (forward primer segment 4) and primer SEQ ID NO: 25 (reverse primer segment 4) one obtains a segment 4 amplicon. If this segment is a genotype II then this amplicon has at least 90% sequence identity with other segments 4 of genotype II ISAV or has less than 85%, or even less than 80% sequence identity with other segments 14 of genotype I ISAV. If this segment is a genotype I then this amplicon has at least 90% sequence identity with other segments 4 of genotype I ISAV or has less than 85%, or even less than 80% sequence identity with other segments 4 of genotype II ISAV.

### Segment 5

For example, using primer SEQ ID NO: 26 (forward primer segment 5) and primer SEQ ID NO: 27 (reverse primer segment 5) one obtains a segment 5 amplicon. If this segment is a genotype II then this amplicon has at least 90% sequence identity with other segments 5 of genotype II ISAV or has less than 85%, or even less than 80% sequence identity with other segments 5 of genotype I ISAV. If this segment is a genotype I then this amplicon has at least 90% sequence identity with other segments 5 of genotype I ISAV or has less than 85%, or even less than 80% sequence identity with other segments 5 of genotype II ISAV.

### Segment 6

For example, using primer SEQ ID NO: 28 (forward primer segment 6) and primer SEQ ID NO: 29 (reverse primer segment 6) one obtains a segment 6 amplicon. If this segment is a genotype II then this amplicon has at least 90% sequence identity with other segments 6 of genotype II ISAV or has less than 85%, or even less than 80% sequence identity with other segments 6 of genotype I ISAV. If this segment is a genotype I then this amplicon has at least 90% sequence identity with other segments 6 of genotype I ISAV or has less than 85%, or even less than 80% sequence identity with other segments 6 of genotype II ISAV.

### Segment 7

For example, using primer SEQ ID NO: 30 (forward primer segment 7) and primer SEQ ID NO: 31 (reverse primer segment 7) one obtains a segment 7 amplicon. If this segment is a genotype II then this amplicon has at least 90% sequence identity with other segments 7 of genotype II ISAV or has less than 85%, or even less than 80% sequence identity with other segments 7 of genotype I ISAV. If this segment is a genotype I then this amplicon has at least 90% sequence identity with other segments 7 of genotype I ISAV or has less than 85%, or even less than 80% sequence identity with other segments 7 of genotype II ISAV.

### Segment 8

For example, using primer SEQ ID NO: 32 (forward primer segment 8) and primer SEQ ID NO: 33 (reverse primer segment 8) one obtains a segment 8 amplicon. If this segment is a genotype II then this amplicon has at least 90% sequence identity with other segments 8 of genotype II ISAV or has less than 85%, or even less than 80% sequence identity with other segments 8 of genotype I ISAV. If this segment is a genotype I then this amplicon has at least 90% sequence identity with other segments 8 of genotype I ISAV or has less than 85%, or even less than 80% sequence identity with other segments 8 of genotype II ISAV.

Another aspect of the invention and/or embodiments thereof, is directed to a vaccine composition comprising a reassorted ISA virus according to any embodiment as described herein.

Suitably the vaccine is used in a treatment or protection of fish against ISA. Suitably the vaccine is used to protect fish against ISA. Suitably the vaccine comprises the reassorted virus of the invention and/or any embodiment thereof, live, killed, attenuated or otherwise. Preferably, the vaccine comprises a killed or attenuated reassorted virus of the invention and/or any embodiment thereof. Preferably, the vaccine comprises an inactivated reassorted ISA virus of the invention and/or any embodiment thereof.

The vaccine may comprise optionally one of more adjuvants and/or pharmaceutically acceptable ingredients. The vaccine of the invention and/or embodiments thereof can be prepared in the form of an aqueous solution, oil- in-water emulsions, or suspension, in a pharmaceutically acceptable vehicle, such as saline solution, phosphate buffered saline (PBS), or any other pharmaceutically acceptable vehicle. The adjuvants may comprise signalling molecules including cytokines, chemokines, immune costimulatory molecules, toll-like receptor agonists or inhibitors of immune suppressive pathways. Also, traditional adjuvants including killed bacteria, bacterial components, such LPS, aluminium salts, oil emulsions, polysaccharide particles, liposomes and biopolymers may be used. Suitable systems use nanoparticles based on biodegradable polymers. Synthetic polymers such as poly(vinylpyridine), polylactide-co-glycolides (PLG) and polylactide-co-glycolide acid (PLGA) may be used.

The pharmaceutically acceptable vehicles that may be used in the formulation of a vaccine of the invention must be sterile and physiologically compatible, e.g. sterile water, saline solution, aqueous buffers such as PBS, alcohols, polyols and suchlike. Said vaccine may also contain other additives, such as adjuvants, stabilisers, antioxidants, preservatives and suchlike. The available adjuvants include, but are not limited to, aluminium salts or gels, carbomers, nonionic block copolymers, tocopherols, muramyl dipeptide, oil emulsions, cytokines, etc. The amount of adjuvant that may be added depends on the nature of the adjuvant. The stabilisers available for use in vaccines according to the invention are, e.g. carbohydrates, including sorbitol, mannitol, dextrin, glucose and proteins such as albumin and casein, and buffers such as alkaline phosphatase. The available preservatives include, among others, thimerosal, merthiolate and gentamicin.

Vaccines comprising inactivated viral antigens often require an immune stimulant for optimal efficacy: an adjuvant. As an excipient, such an adjuvant needs to be pharmaceutically acceptable, and cost effective. Well known adjuvants used in fish vaccines are: aluminium salts, liposomes, glucans, alginate, and in particular: oils.

The vaccine of the invention and/or embodiments thereof may be prepared using conventional methods known by a person skilled in the art. In a embodiment, said vaccine is prepared by providing the reassorted ISA virus of the invention and/or any embodiments thereof and then mixing it with oil to form an emulsion. The ISA virus is preferably inactivated. In a suitable embodiment, the ISA virus culture is used. Suitably the ISA virus culture may be concentrated or diluted depending on the titre of the virus and the dose required. Alternatively, the ISA virus is first purified from the culture medium. The ISA virus is suitably mixed with an oil to form an emulsion. Suitable emulsions are water-in-oil (W/O) emulsions, oil-in-water (O/W) emulsions, and water-in-oil-in-water emulsions (W/O/W) and microemulsions. Suitable oils are mineral oils, non-mineral oils and synthetic oils. Additional adjuvants may be added such as Toll receptors ligands and cytokines.

To generate and maintain such an emulsion requires the input of both mechanical as well as chemical energy: the separate liquids are mixed in an appropriate device using certain levels of shear-force, pressure, and temperature to disperse one phase into another. The chemical energy is provided using an emulsifier (also: surfactant) which stabilises the dispersed phase by taking position at the interphase of water and oil. A vaccine emulsion can be made up of one or more adjuvants, with one or more emulsifiers.

A large number of emulsifiers for use in emulsion vaccines is available, and more are constantly being developed. A short review of this field was made by Ascarateil & Dupuis (2006, Vaccine vol. 24S2, p. S2/83 - S2/85).

Examples of combinations of adjuvants and emulsifiers as used in commercial veterinary vaccines are: Amphigen® (Zoetis), containing a light mineral oil with lecithin as emulsifier; Xsolve® (previously called: Microsol-Diluvac Forte®, MSD Animal health), which contains a combination of the adjuvants light mineral oil and vitamin E-acetate, with the emulsifier Tween® 80 (Polysorbate 80, or polyoxyethylene sorbitan mono-oleate); and MetaStim® (Zoetis), comprising squalane, Pluronic® (a non-ionic tri-block copolymer of blocks of polyoxyethylene and polyoxypropylene), and Tween 80.

The vaccine of the present invention and/or embodiments thereof may comprise other fish pathogens selected from the group consisting of salmon alpha virus (SAV), Infectious Haematopoietic Necrosis virus (IHNV),Tilapia Lake virus (TLV), Piscine Reovirus (PRV), Cardiomyopathy Virus (CMV), Epizootic Hematopoietic Necrosis virus (EHNV), Piscirickettsia, Franciscella, Aeromonas, Vibrio, Moritella, Edwardsiella, Flexibacter, Pasteurella, Cytophaga, Coryne pathogen, Renia pathogen, Arthrobacter, Flavoa pathogen, Fusarium, Bacillus, Yersinia, Myco pathogen, Neorickettsia, Listonella, Flexibacter, Streptococcus, Shewanella, Pseudomonas, Photo pathogen, Clostridium, Tenacibaculum, Lactococcus, Leucothrix, and Nocardia.

Another aspect of the invention and/or embodiments thereof, is directed to a method to produce reassorted ISA virus according to any embodiment as described herein comprising the steps of (i) infecting a culture host with an ISA virus strain of genotype I and an ISA virus strain of genotype II;
(ii) culturing the culture host in order to produce reassorted ISAV virus and
(iii) determine if the reassorted ISAV virus has a segment 6 of genotype II present and at least one other segment of genotype I.

A reassorted ISA virus can be made by coculturing ISA virus of Genotype I and ISA virus of Genotype II. For Influenza A viruses it was known that they have the unique capacity to undergo a high degree of antigenic variation within a short period of time. Considerable variation occurs among the HA and neuraminidase (NA) antigens of influenza A viruses. In the case of ISAV isolates, there is no information about antigenic variation (Kibbenge 2001 J. Gen. Virol 82 ). However, it appears that in order to obtain a reassorted ISA virus the same methods to make reassorted Influenza A viruses are suitable. After the co-culture ISA virus with segment 6 from genotype ll are selected. PCR can be used to select a virus with segment 6 from genotype II. Primers and probes as disclosed in Kibenge et al. 2001, J. Gen Virol, 82, p2869-2879; Kibenge et al. 2016 Virology Journal 13:3; or Cottet et al. J. Virol, 2010, p11916-11928, can be used as well as the primers used in the experimental section as described herein. The viruses are co-cultured in adherent cells culture or in suspension cell culture. Preferably viruses are co-cultured in adherent cells culture. Suitable the culture host are CHSE-214 cells. Preferably, the viruses are co-cultured in serum free medium.

In one embodiment of the invention and/or embodiments thereof, the method to make reassorted ISA virus comprises the further step of (iv) purifying the virus obtained in step (iii). The virus thus obtained may also be further cultured on suspension cells and/or serum free medium. The culturing on suspension cells and/or serum free medium can also be used to select for a reassorted virus according to the invention and/or any embodiment thereof.

Another aspect of the invention and/or embodiments thereof, is directed to a method to culture reassorted ISA comprising
(i) infecting a culture host with the reassorted virus according to any embodiment as described herein
(ii) culturing the host from step (i) to produce further virus.

Preferably the reassorted ISA viruses are cultured on suspension culture host. Preferably the reassorted ISA viruses are cultured in serum free media. Suitably the culture host are suspension CHSE-214 cells. Preferably the reassorted ISA are cultured in serum free media on suspension CHSE-214 cells.

Fish suspension cells are well known to the skilled person. Adherent cells such as many fish cells, can become suspension cells when grown in a suspension culture medium. Cells adhering to the container wall are treated with a mixed solution of trypsin and EDTA according to a conventional method and detached. After obtaining floating cells in this manner, the cells are cultured while being acclimated to a suspension culture medium. Suspension culture medium is commercially available, such as Eagle MEM medium for suspension culture and Joklik’s modified Eagle medium.

JPH1066563 discloses producing a fish-derived suspension culture cell line in this way. A carp epithelioma-derived suspension culture cell line (EPC / NIAH cell) and a Japanese eel kidney-derived suspension culture cell line (EK-1 / NIAH cell) were developed.

Fish cells like salmon CHSE cells and carp FHMP cells can be grown in a serum free medium by repeating and selecting cells that grow well in a medium with increasingly less serum, until there is no serum present in the medium.

JP2003219873 discloses that the suspensions CHSE-214 cells can be cultured in serum free medium and that they can be saved at low temperature such as about 4° C.

Shea and Berry (In Vitro vol 19, no. 11, (1983), p818-825) discloses that a range of fish cell lines can be grown on serum free medium, including CHSE-214 cells and were able to support the replication of gold-fish virus-2 at levels equivalent to cells grown in medium with serum.

JP2003219873 discloses CHSE-214 cells that grow in suspension and can be cultured serum-free medium. Also, other fish cells have been reported capable of growing in suspension. One is a method of culturing CHSE-214 cells in a suspended EMEM-S culture solution containing carboxymethyl cellulose (Lidgerding, Develop. Biol. Standard, 49, 233-241 (1981)), and the other is a method using the MB752 / 1 medium of Waymouse without CaCl2 ( Hasobe M., et al., Bull. Eur. Asso. Fish Pathol., 11, 142-144 (1991)).

JP2003219873 discloses that the CHSE-214 suspension cells can be cultured in serum free culture with addition of lactalbumine hydrolysate as serum substitute. A suitable medium is Waymouth medium MB752 / 1 without addition of CaCl2, 14 mM Hepes (pH adjustment with NaOH), 10% lactalbumin hydrolysate aqueous solution 5% and kanamycin 0.06 g / L added. The medium has a pH of 7.4.

Serum free medium is commercially available. As substituent for serum or albumin, yeast extracts or wheat gluten hydrolasates may be used. WO99/57246 discloses serum free cell culture medium completely devoid of animal proteins and lipids. In here the protein is derived from rice, soy, potato, corn and aloe vera and the lipid is derived from rice, soy, potato, corn and aloe vera as well as from bacteria, yeast and fungi.

In one embodiment of the invention and/or embodiments thereof, the method to grow a reassorted ISA virus further comprises the step (iii) purifying virus obtained in step (ii).

Another aspect of the invention and/or embodiments thereof, is directed to a reassorted ISA virus according to the invention and/or any embodiment thereof for use in a method to vaccinate fish against ISA virus . Another aspect of the invention and/or embodiments thereof, is directed to a reassorted ISA virus according to the invention and/or any embodiment thereof for use in a method to protect fish against infection of an ISA virus . Another aspect of the invention and/or embodiments thereof, is directed to a method of treatment to protect against infection of an ISAV virus by administering the reassorted ISA virus according to the invention and/or any embodiment thereof

In a preferred embodiment of the invention and embodiments thereof, the reassorted virus provides protection against infection of an ISA virus of genotype I. In a preferred embodiment of the invention and embodiments thereof, the reassorted virus provides protection against infection of an ISA virus of genotype II. In a preferred embodiment of the invention and embodiments thereof, the reassorted virus provides protection against infection of an ISA virus of genotype I and Genotype II. Advantageously the reassorted ISA virus of the invention and/or any embodiments thereof provides protection and can be used to treat an infection of ISA virus of Genotype I and of Genotype II.

Suitably, the fish is a salmonid. Salmonids include salmon, trout, chars, freshwater whitefishes, and graylings. Suitably, the fish is a salmon, trout, or char. Suitably, the fish is a salmon or trout. Suitably, the fish is a salmon and most suitably Atlantic salmon (Salmo salar).

As used herein, the terms “protecting” or “providing protection to” and “aids in the protection” do not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection. Protecting against ISAV means that there is a reduction in at least one of the symptoms selected from the group consisting of severe anaemia, ascites, haemorrhage in internal organs and darkening of the liver. Protecting against ISAV may also mean that there is a reduction in mortality after infection with ISAV.

Preferably there is at least a 10% reduction in at least one of the symptoms selected from the group consisting of severe anaemia, ascites, haemorrhage in internal organs and darkening of the liver.. More preferably, there is at least 20%, 25% 30%, 40%, 50%, 60%, 70% 75%, 80%, 85%, 90%, or even at 95% reduction in at least one of the symptoms selected from the group consisting of severe anaemia, ascites, haemorrhage in internal organs and darkening of the liver. compared to a fish that has been infected with ISAV and not treated with the reassorted virus of the present invention and/or embodiments thereof.

Suitably protection means that there is a reduction in mortality of at least 10% when compared to fish that are infected with ISAV and have not been treated with the resorted ISA virus of the present invention and/or any embodiment thereof. More preferably, there is at least 20%, 25% 30%, 40%, 50%, 60%, 70% 75%, 80%, 85%, 90%, or even at 95% reduction in mortality when compared to fish that are infected with ISAV and have not been treated with the resorted ISA virus of the present invention and/or any embodiment thereof. Suitably there is a reduction in mortality of between 100% and 20%, more preferably there is a reduction in mortality of between 30% and 90%, more preferably there is a reduction in mortality of between 35% and 85%, more preferably there is a reduction in mortality of between 40% and 80%, more preferably there is a reduction in mortality of between 45% and 75%, more preferably there is a reduction in mortality of between 50% and 70%, more preferably there is a reduction in mortality of between 55% and 65%.

The “administration” of the vaccine according to the invention to a fish target can be performed using any feasible method and route. Typically, the optimal way of administration will be determined by the type of the vaccine applied, and the characteristics of the target and the bacterial disease that it is intended to protect against. Depending on whether the vaccine according to the invention is based on an O/W or on a W/O emulsion, different techniques of administration can be applied. For example as an O/W emulsion vaccine the vaccine according to the invention can be administered by enteral or mucosal route, i.e. via eye drop, nose drop, oral, enteric, oro-nasal drop, spray. Other possibility is via a method of mass administration, such as via drinking water, coarse spray, atomisation, on-feed, etcetera.

Preferred way of administration for a method of vaccination according to the invention is by parenteral route. “Parenteral” refers to administration through the skin, for example by intramuscular, intraperitoneal, intradermal, submucosal, or subcutaneous route.

The volume of a dose of the vaccine according to the invention and/or embodiments thereof, e.g. when administered by parenteral route, is a volume that is acceptable for the target fish, and can for instance be between about 0.01 ml and about 5 ml. Preferably one dose is a volume between 0.02 ml and 2 ml, more preferably one dose is between 0.05 ml and 1 ml, more preferably between 0.1 ml and 0.5 ml.

When administered by intramuscular route, the volume of one dose is preferably between about 0.05 and about 2 ml, more preferably between 0.1 and 1 ml.

In an embodiment, the vaccine comprises per dose between 5 and 500 HA units of ISA virus, more preferably between 10 and 200 HA units of ISA virus, more preferably between 15 and 150 HA units, more prerably between 20 and 100 HA units, more preferably between 25 and 90 HA units, more preferably between 30 and 85 HA units, more preferably between 35 and 80 HA units, more preferably between 40 and 75 HA units, more preferably between 45 and 70 HA units, more preferably between 50 and 65 HA units, and more preferably between 55 and 60 HA units.

HA units (HAU) are hemagglutination units and can be determined by well known methods. One such method is measuring the amount of agglutination of virus to red blood cells (RBC) in a serial dilution. The HAU can be determined by measuring the OD₄₉₂. The hemagglutinating (HA) titre is calculated at OD₄₉₂ = 1.5. If none of the dilutions used in the titration give an OD of exactly 1.5 the titre is found by interpolation between the highest dilution with OD<1.5 and the lowest dilution giving an OD>1.5 :

$$\text{Titre at OD1} \text{.50} \left(\text{TOD1} \text{.5}\right) = \left(1.5 - \text{b}\right) / \text{a}$$

where

$$\text{a} \text{=} \left(\text{Y2} \text{-} \text{Y1}\right) / \left(\text{X2} \text{-} \text{X1}\right)$$

$$\text{b} \text{=} \text{Y1} \text{} \text{-} \text{a} \text{*} \text{X1}$$

X1 : Lowest dilution giving an OD > 1.5
Y1 : OD at X1 (T>1.5)
X2 : Highest dilution giving an OD < 1.5
Y2 : OD at X2 (T<1.5)

The final HAU/ml concentration of the sample is expressed relative to the reference standard of predetermined value (4000 HAU/ml) following the formulae:

$$\begin{matrix}\text{Sample concentration} \left(\text{HAU} / \text{ml}\right) = \\ \text{} \left[\frac{\text{mean HAU} / {\text{ml}}_{\text{Sample}}}{\text{mean HAU} / {\text{ml}}_{\text{Reference}}}\right] \times \text{Reference concentration}\end{matrix}$$

The invention will now be further described by the following, non-limiting, examples.

### EXAMPLES

### Isolation of ISAV

ISAV isolates of Norwegian, Chilean and Canadian origin were cultured in adherent CHSE-214 cells and screened (indirectly) for increased replication levels by real-time PCR. Candidate ISAV cultures with high replication levels were subject to limiting dilution and plaque purification to obtain single ISAV clones.

TABLE 1

Summary table of origin and genotype of ISAV isolates used for culturing.

| | | |
|---|---|---|
| ISA isolate | Origin Country (County), Year | Genotype |
| Misund 99 | Norway (Sogn og Fjordane), 1999 | I |
| Fjaler 00 | Norway (Sogn og Fjordane), 2000 | I |
| Eikelandsosen 87 | Norway (Hordaland), 1987 | I |
| Bremnes 98 | Norway (Hordaland), 1998 | I |
| Canada 97 (Bay of Fundy) | Canada (New Brunswick), 1997 | II |
| CH 0108 | Chile (not specified), 2008 | I |
| CH 3509 | Chile (Region X), 2009 | I |
| CH 107-12 | Chile (Region X, Quellón), 2008 | I |

Isolates were first isolated and passaged once in ASK-3 cells before inoculation on 25 cm² cell culture flasks containing an 80% confluent CHSE-214 cell layer supplemented with 2% FCS maintenance EMEM medium.

In general, the real-time PCR data showed that ISA isolates of the European genotype showed little or no signs of adapting to increased replication in CHSE-214 adherent cells, and in particular the Chilean isolates were found not possible to maintain through serial passages in CHSE-214 cells. On the other hand, the Canadian isolate (Bay of Fundy -97) seemed to adapt well to replication in CHSE-214 cells as indicated by the real-time RT PCR Ct-values. The Canada-97 isolate belongs to the North-American ISAV genotype, which is known to give poor protection against challenge with ISAV of European genotype.

In addition, two separate cell culture flasks were concomitantly infected with a mix of ISAV (see Table 2). For each passage, virus supernatant was inoculated on separate cell culture flasks every 12-14 days at a volume ratio 1:5 or 1:100. At passage 6, two of the cell cultures (no. 5 and 8) were split into two lineages; - one passaged further at 1:5 ratio (lineages 5 and 8) and one at a ratio 1:100 (lineages 10 and 11; see Table 2).

TABLE 2

Overview of the different virus cultures/lineages, with real-time RT-PCR results from selected cell-culture passages. Virus cultures/lineages 1-9 were passaged at a ratio 1:5, whereas 10 and 11 were passaged at a ratio 1:100. Virus culture/lineage 10 and 11 were derived from no 5 and 8 from passage level 6 onwards. Term= terminated; undetec=undetected

| ISAV culture /lineage no. | | Ct value in ASK-3 (p1) | Ct-value in CHSE-214 | | | |
|---|---|---|---|---|---|---|
| | | | p5 | p7 | p15 | p20 |
| 1 | Misund 99 | 21.5 | 32.4 | 29.1 | 27.7 | 27.8 |
| 2 | Fjaler 00 | 21.9 | 35.2 | terminated | - | - |
| 3 | Eikelandsosen 87 | 21.1 | 31.5 | 25.8 | 28.5 | 29.9 |
| 4 | Bremnes 98 | 21.0 | undetec | term | - | - |
| 5 | Canada 97 | 23.3 | 32.2 | 25.2 | term | |
| 6 | CH 0108 | 20.2 | 37.1 | term | - | - |
| 7 | CH 3509 | 18.9 | undetec | term | - | - |
| 8 | CH 107-12/Canada 97 (mix) | 22.4 | 28.3 | 23.0 | 23.9 | 23.3 |
| 9 | Mix of no 1-8 | - | 30.7 | 29.1 | 22.8 | 24.5 |
| 10 | Canada 97 (1:100) | - | - | 24.6 | 25.1 | term |
| 11 | CH 107-12/Canada 97 (mix) (1:100) | - | - | 22.8 | 22.2 | 23.3 |

Based on the real-time PCR results we considered the three mixed infection flasks (8, 9 and 11 (table 2)) to be the most promising for use in a CHSE-214 -based production system, since these appeared to have adapted well to growth in CHSE-214 cells. To test if the virus in these lineages also would protect against ISA, a preliminary efficacy study was performed which demonstrated that a test vaccine formulated with inactivated virus from the mixed infection CH 107-12/Canada 97 (no 11, table 2) provided excellent protection against challenge with ISAV of Chilean origin.

### Screening of ISAV Passages in CHSE-214 Adherent Cells

Since the virus material from the selected lineage above (CH 107-12/Canada 97 (no 11, table 2)) was created from a concurrent infection with two different genotypes of ISAV, single viral clones were purified and test ed again for high replication in CHSE-214 cells as well as efficacy for giving protection against infection with ISAV of Chilean origin.

For every passage prior to plaque purification, cell culture material (supernatant+cell layer) was sampled and frozen at -80° C. The frozen material was subjected to RNA extraction (100 µl) and subsequent real time RT-PCR to test for the presence of ISAV RNA using an in-house TaqMan MGB-assay directed towards genomic segment 8. Low Ct values indicate high replication and successful propagation of ISAV.

Following limiting dilution and plaque purification, the identity of single clones was checked using both Chile- and Canada-specific real-time RT-PCR assays targeting ISAV genomic segments 5 and 6.

Using 4 µl RNA template, ISAV genotype specific real time RT-PCR assays were performed on an ABI 7500 Fast PCR machine (Applied Biosystems) for both genomic segment 5 (encoding the fusion protein) and for genomic segment 6 (encoding the hemagglutinin-esterase) based on the following Taqman MGB assays (designed with the program Primer Express 3.0.1, Applied Biosystems) which are specific for the Chilean/European and Canadian/North-American genotypes:

Primers and Taqman MGB probe sequences for genotype specific detection of ISAV genomic segments 5 and 6:

| ISAV segment 5 | Chilean/European seg5 assay | Canadian/North-American seg5 assay |
|---|---|---|
| Forward primer seq. | SEQ ID NO: 1 CGGCCTCGCTAACCAACAT | SEQ ID NO: 4 GAGTCGGAGGAGCGTGGTT |
| Reverse primer seq. | SEQ ID NO: 2 CCCAAAGTAAATCCACAGC ATTT | SEQ ID NO: 5 CGGCACCGTCACCTTGA |
| Taqman MGB Probe seq. (FAM labelled) | SEQ ID NO: 3 ATGGTCTAAATACAACTTC | SEQ ID NO: 6 CAAGCCTACTTAAATGGAA |

| | | |
|---|---|---|
| ISAV segment 6 | Chilean/European seg6 assay | Canadian/North-American seg6 assay |
| Forward primer seq. | SEQ ID NO: 7 AAAATCAGGGTAGACGCA ATCC | SEQ ID NO: 10 CTGCATTGCTGCCTCTTCTG |
| Reverse primer seq. | SEQ ID NO: 8 GAACAGAGCAATCCCAAA ACCT | SEQ ID NO: 11 CCGCATCCGTTCACCAA |
| Taqman MGB Probe seq. (FAM labelled) | SEQ ID NO: 9 ACCTCAGCTGAACCAA | SEQ ID NO: 12 AAACGTAGGCGGAGTCA |

The sequences of primers and probe used for the real time RT-PCR assay specific for EIV HA (exogenous internal control) are those published by Foord et al: (Foord AJ, Selleck P, Colling A, Klippel J, Middleton D, Heine HG. Real-time RT-PCR for detection of equine influenza and evaluation using samples from horses infected with A/equine/Sydney/2007 (H3N8).; Vet Microbiol. 2009 May 28; 137(1-2):1-9)

| | |
|---|---|
| EIV HA assay | |
| Forward primer seq. | SEQ ID NO: 13 CAGCGCTTTCAGCAATTGC |
| Reverse primer seq. | SEQ ID NO: 14 GAYCGGAGCGATGCATAGTC |
| Taqman MGB Probe seq. (VIC labelled) | SEQ ID NO: 15 CCCATATGACATCCC |

More than 200 clones from the plaque purification assay were tested for the combination of genomic segments 5 and 6. Of these, 4 different clones were selected (Table 3) based on a combination of Chilean and Canadian genomic segments 5 and 6.

TABLE 3

Real-time RT-PCR genotyping of genomic segments 5 and 6 of ISAV clones 4, 22, 77 and 93. All 4 clones were derived from virus material harvested from ISAV culture/lineage no 11, either from passage level 15 or 20.. Additional 5 passages in CHSE-214 cell culture were performed including steps involving limiting dilution and plaque purification

| ISAV clone | Ge omic segment 5 | Genomic segment 6 | Virus material | |
|---|---|---|---|---|
| | | | ISAV culture/lineage no | Passage level* |
| Clone 4 | II | II | 11 | P25 [P20 + 5] |
| Clone 22 | I | II | 11 | P25 [P20 + 5] |
| Clone 77 | I | II | 11 | P20 [P15 + 5] |
| Clone 93 | I | I | 11 | P20 [P15 + 5] |

* Final passage level subject to genotyping, with number of passages before and after plaque purification.

The clones in Table 3 were tested for replication in CHSE-214 cells, CHSE-214 suspension cells. antigen (HA) yield, as well as in efficacy trials following formulation, vaccination and challenge with ISAV.

### Growing in CHSE 214 Suspension Cells

The clones of table 3 were tested for replication in CHSE 214 adherent cells and CHSE 214 suspension cells.

CHSE-214 adherent cells were seeded in a culture flask or roller bottle at concentration of 8×10⁴ cells/cm² in culture medium and incubated at at 20° C. Cells were passed every 6-8 days. Harvest of the virus culture from roller bottles and culture flask was done using glass pearls to include all cells in the harvest.

A bioreactor contains CHSE-214 suspension cells and serum free medium, was infected with ISAV with a MOI of 0.01 and incubated at 14° C. After 14-20 days the virus was harvested.

| | | | | |
|---|---|---|---|---|
| ISAV clone | Genomic segment 5 | Genomic segment 6 | Growth on CHSE-214 adherent | Growth on CHSE-214 suspension |
| Clone 4 | II | II | Y | Y |
| Clone 22 | I | II | Y | Y |
| Clone 77 | I | II | Y | Y |
| Clone 93 | I | I | Y | N |

Only clones with Genomic segment 6 from North America, Genotype II, were able to grow on suspension cells.

### Vaccine Efficacy

Twelve groups (35 fish/group) of Atlantic salmon pre-smolts were IP vaccinated either with different formulations containing an ISA-component or with saline (negative control)..The fish were kept in two 500 L freshwater tanks (one tank with 5 test groups, one negative control group and one positive control group (vaccine based on Bremnes 98 antigen (Norwegian isolate), and a second tank with 4 test groups, one negative control group and two positive control groups (vaccines based on Bremnes 98 antigen)) during 6 weeks until challenge by cohabitation with ISAV (15% shedders). Mortality was recorded daily for 49 days following challenge. Quantification of the ISA antigens was done by hemagglutination titration test and/or antigenic mass ELISA test following standard procedures.

### Vaccine Compositions

Water in oil formulations (45/55) with adjuvant (mineral oil; inactivated whole ISAV, 50 µl per dose, see table 4

TABLE 4

Vaccine composition

| vaccine | Antigen strain | Genomic segment 5 | Genomic segment 6 | Virus material | |
|---|---|---|---|---|---|
| | | | | ISAV culture | Dose HA U |
| 1 | Clone 4 | II | II | CHSE-214 suspension | 107 |
| 2 | Clone 4 | II | II | CHSE-214 suspension | 40 |
| 3 | Clone 22 | I | II | CHSE-214 suspension | 107 |
| 4 | Clone 22 | I | II | CHSE-214 suspension | 40 |
| 5 | Clone 77 | II | II | CHSE-214 suspension | 110 |
| 6 | Clone 77 | I | II | CHSE-214 suspension | 40 |
| 7 | Clone 93 | I | I | CHSE-214 adherent t | 85 |
| 8 | Clone 93 | I | I | CHSE-214 adherent t | 40 |
| 9 | Norwegian isolate Eikelandsos en H1/87 | I | I | CHSE-214 suspension | 0 |
| Control 1 | Norwegian isolate Bremnes-98 | I | I | SHK-1 adherent cells | 89 |
| Control 2 | Norwegian isolate Bremnes-98 | I | I | SHK-1 adherent cells | 52 |
| Control 3 | saline | | | | |

Challenge material was ISAV Chilean strain CH0108 180509 given at 1.5×10² TClD₅₀ /fish 0.05 ml dose, intramuscular.

### Fish, Atlantic Salmon, 30-35 Grams, at 245 Fish + 44 Shedders Per Tank

Vaccine was given intraperitoneally, 0.05 ml dose per fish. Fish were vaccinated and 6 weeks later, challenged by introducing of shedder fish.

Mortality were recorded daily for a period of 49 days post challenge (endpoint), and plotted separately as cumulative percent mortality. Vaccine efficacy is expressed as the elative percent survival (RPS) using saline injected fish as control group, and was calculated as RPS_{EP}, relative percentage survival in vaccinates at endpoint·
RPS= (1-M_{vacc}/M_{contr})×100
M_{vacc}= cumulative mortality vaccinated group
M_{contr} = cumulative mortality control group

### RESULTS

The challenge experiment was successful, obtaining close to 100% mortality (high/very high challenge in control groups group. Autopsied dead or moribund fish showed clinical signs and gross pathology consistent with infection ISA V.

Survival data for each group is shown in Table 5

All vaccination groups showed a highly significant protection against ISA, except the Eikelandsosen-group which had an RPS_{EP} of 21% (Fisher exact / > , =0.275). Groups vaccinated with either ISA clone 77 or 93 showed the highest protection (100% RPS) in the 40 HAU/dose groups.

TABLE 5

Summary of mortality data in Tank 1 and 2 for control group and test vaccines
Tank 1

| Group | Vaccine ISA antigen | ISA dose HA (EU) | Dead/Live | RPS_{EP} | Fisher test p value compared to saline |
|---|---|---|---|---|---|
| 1 | Clone 4 | 107 | 5/30 | 85 | P<0.0001 |
| 2 | Clone 4 | 40 | 2/33 | 94 | P<0.0001 |
| 3 | Clone 22 | 89 | 0/35 | 100 | P<0.0001 |
| 4 | Clone 22 | 40 | 3/32 | 91 | P<0.0001 |
| 5 | Eikelandsosen | | 27/8 | 21 | P<0.05 |
| 6 | Bremnes-98 | (12.5) | 2/33 | 94 | P<0.0001 |
| 7 | Saline | | 34/1 | | |
| Tank 2 | | | | | |
| Group | Vaccine ISA antigen | ISA dose HA (EU) | Dead/Live | RPS_{EP} % | Fisher test p value compared to saline |
| 8 | Clone 77 | 110 | 1/34 | 96 | P<0.0001 |
| 9 | Clone 4 | 40 | 0/35 | 100 | P<0.0001 |
| 10 | Clone 22 | 85 | 0/35 | 100 | P<0.0001 |
| 11 | Clone 22 | 40 | 0/35 | 100 | P<0.0001 |
| 12 | Bremnes-98 | 52.5 | 1/34 | 96 | P<0.05 |
| 13 | Bremnes-98 | (12.5) | 2/33 | 92 | P<0.0001 |
| 14 | Saline | | 25/10 | | |

### Genotyping Clone 77

The genotyping and characterization of all clone 77 is summarized in Table 6 below. This shows that clone 77 is a novel reassorted ISA virus resulting from genomic segment mixing following co-infection of CHSE-214 cells with the two ISAV isolates CH 107-12 and Canada 97.

Table 6. Overview of the genotypes I or II determined for each of the8 genomic RNA segments of clone 77.

| Segment | Encoded protein | Genotype |
|---|---|---|
| 1 | Polymerase (PB2) | II |
| 2 | Polymerase (PB1) | II |
| 3 | Nucleoprotein (NP) | II |
| 4 | RNA polymerase (PA) | II |
| 5 | Fusion (F) | I |
| 6 | Hemagglutinin-esterase (HE) | II |
| 7 | nonstructural proteins (NS1, NS2) | II |
| 8 | Matrix proteins (M1, M2) | I |

Table 7 PCR primer sets used to amplify parts of the 8 genomic segments of clone 77 and corresponding amplicon size.

| Segment | Forward primer (name/sequence) | Reverse primer (name/sequence) | Amplicon size |
|---|---|---|---|
| 1 | SEQ ID NO: 16 GTAATGCACCTAAAGGGCAC | SEQ ID NO: 17 CCTCCTTCACTYCTTACTGRAA | 583 bp |
| 2 | SEQ ID NO: 18 GCAACAGGTTTCAGRGGAAG A | SEQ ID NO: 19 GTRAACACTGCTCTCGGCT | 245 bp |
| 3 | SEQ ID NO: 20 YATGAGGAACTGCGGAGGAG | SEQ ID NO: 21 GCTGTTGCAACCATTGACAC | 438 bp |
| 4 | SEQ ID NO: 22 TACACAGRGCGACMACAACS | SEQ ID NO: 23 CAYRCTYCCCACAACACCCC | 500 bp |
| 4 | SEQ ID NO: 24 ACACCGATCAGCACATG | SEQ ID NO: 25 TGGCCCTCCTCTGTAAGGAA | 228 bp |
| 5 | SEQ ID NO: 26 GGAGTGCTCCTGGAGGTGTA | SEQ ID NO: 27 CCTCTGGTGGACATCCTCTG | 366 bp |
| 6 | SEQ ID NO: 28 AGCGCTGAAGTATCCGCATC CGTTCACCAAGTTG | SEQ ID NO: 29 ACCAACCTTCGTGGGAGCAGC TGAGC | 211 bp |
| 7 | SEQ ID NO: 30 GYCTTACTGARAYGGGRTCA | SEQ ID NO: 31 TGAGRTASCAKCCKCCCCAG | 450 bp |
| 8 | SEQ ID NO: 32 CGACGATGACTCTCTACTGT GTGAT | SEQ ID NO: 33 TGCATCCTGCTGYGYAGCAT | 223 bp |

1. A reassorted Infectious salmon anemia (ISA) virus comprising Genome segments 1-8 wherein at least one genome segment is from Genotype I and at least one genome segment is from genotype II, and wherein genome segment 6 is of Genotype II.

2. The reassorted ISA virus of claim 1, wherein at two least segments are of Genotype I.

3. The reassorted ISA virus of claim 1, wherein genome segment 5 is of Genotype I.

4. The reassorted ISA virus of claim 1, wherein genome segment 8 is of Genotype I.

5. The reassorted ISA virus of claim 1, wherein the genome segment of Genotype I is of Chilean origin.

6. The reassorted ISA virus of claim 1, wherein the segment of Genotype II is of Canadian origin.

7. A vaccine composition comprising the reasserted ISA virus of claim 1.

8. A method of producing the reasserted ISA virus of claim 1 comprising the steps of:
(i) infecting a culture host with an ISA virus strain of genotype I and an ISA virus strain of genotype II;
(ii) culturing the culture host in order to produce reassorted ISAV virus; and
(iii) determining whether the reassorted ISAV virus has a segment 6 of genotype II present and at least one other segment of genotype I.

9. The method of claim 8, further comprising a step of (iv) purifying the virus obtained in step (iii).

10. A method of culturing a reassorted ISA virus comprising
(i) infecting a culture host with the reassorted virus of claim 1; and
(ii) culturing the host from step (i) to produce further virus.

11. The method of claim 10, further comprising a step of (iii) purifying virus obtained in step (ii).

12. The method of claim 8, wherein the culture host is a suspension cell.

13. The method of claim 8, wherein the culture host is a CHSE-214 cell.

14. The method of claim 8, wherein the ISA virus is cultured in serum free medium.

15. (canceled)

16. The reassorted ISA virus of claim 1, wherein segment 6 is of genotype II when the sequence of an amplicon obtained by forward primer of SEQ ID NO: 10 and reverse primer SEQ ID NO: 11 has at least 90% sequence identity with a segment 6 of NC_006499.

17. The reassorted ISA virus of claim 1, wherein the genotype of a segment is determined by obtaining an amplicon of a segment by the following primer pairs:

| Segment | Forward primer | Reverse primer |
|---|---|---|
| 1 | SEQ ID NO: 16 | SEQ ID NO: 17 |
| 2 | SEQ ID NO: 18 | SEQ ID NO: 19 |
| 3 | SEQ ID NO: 20 | SEQ ID NO: 21 |
| 4 | SEQ ID NO: 22 | SEQ ID NO: 23 |
| 4 | SEQ ID NO: 24 | SEQ ID NO: 25 |
| 5 | SEQ ID NO: 26 | SEQ ID NO: 27 |
| 6 | SEQ ID NO: 28 | SEQ ID NO: 29 |
| 7 | SEQ ID NO: 30 | SEQ ID NO: 31 |
| 8 | SEQ ID NO: 32 | SEQ ID NO: 33 |

and comparing the sequence of the amplicon with the sequence of corresponding segment of NC_006499;
wherein when the amplicon has less than 80% sequence identity with the corresponding segment of NC_006499 then the segment is a genotype I; and
wherein when the amplicon has at least 90% sequence identity with the corresponding segment of NC_006499 then the segment is a genotype II.

18. The reassorted ISA virus of claim 17, wherein when the amplicon has less than 85% sequence identity with the corresponding segment of NC_006499 then the segment is a genotype I; and
wherein when the amplicon has at least 90% sequence identity with the corresponding segment of NC_006499 then the segment is a genotype II.

19. The method of claim 8, wherein segment 6 is of genotype II when the sequence of an amplicon obtained by forward primer of SEQ ID NO: 10 and reverse primer SEQ ID NO: 11 has at least 90% sequence identity with a segment 6 of NC_006499.

20. The method of claim 8, wherein the genotype of a segment is determined by obtaining an amplicon of a segment by the following primer pairs:

| Segment | Forward primer | Reverse primer |
|---|---|---|
| 1 | SEQ ID NO: 16 | SEQ ID NO: 17 |
| 2 | SEQ ID NO: 18 | SEQ ID NO: 19 |
| 3 | SEQ ID NO: 20 | SEQ ID NO: 21 |
| 4 | SEQ ID NO: 22 | SEQ ID NO: 23 |
| 4 | SEQ ID NO: 24 | SEQ ID NO: 25 |
| 5 | SEQ ID NO: 26 | SEQ ID NO: 27 |
| 6 | SEQ ID NO: 28 | SEQ ID NO: 29 |
| 7 | SEQ ID NO: 30 | SEQ IDNO: 31 |
| 8 | SEQ ID NO: 32 | SEQ ID NO: 33 |

and comparing the sequence of the amplicon with the sequence of corresponding segment of NC_006499;
wherein when the amplicon has less than 80% sequence identity with the corresponding segment of NC_006499 then the segment is a genotype I; and
wherein when the amplicon has at least 90% sequence identity with the corresponding segment of NC_006499 then the segment is a genotype II.

21. A method of vaccinating a fish against an ISA virus comprising administering to the fish the reassorted ISA virus of claim 1, or a vaccine comprising said reassorted ISA virus.
